(19) United States
(12) Patent Application Publication
(10) Pub. No.: US 2013/0035631 A1
(43) Pub. Date: Feb. 7, 2013

(54) Cannula Device and Method and Device for Providing a Cannula Device with an Implant

(71) Applicants: Heiko Spilgies, Munich (DE); Andreas Mueller, Taufkirchen (DE); Franz Schoenbach, Fischbachau (DE)

(72) Inventors: Heiko Spilgies, Munich (DE); Andreas Mueller, Taufkirchen (DE); Franz Schoenbach, Fischbachau (DE)

(73) Assignee: ACINO AG, Miesbach (DE)

(21) Appl. No.: 13/640,769

(22) PCT Filed: Mar. 9, 2011
(86) PCT No.: PCT/EP11/01161

§ 371 Date: Oct. 12, 2012

(30) Foreign Application Priority Data
Apr. 13, 2010 (DE) 102010014791.5

(51) Int. Cl. A61M 37/00; B29C 47/02

(52) U.S. Cl. 604/57; 425/113; 264/267

(57) ABSTRACT

To provide a cannula device (4) with an implant (3), the cannula device (4) is held at an extrusion die in such a way that the cannula device (4) is in contact with a sealing surface of the extrusion die. A flowable implant material is conveyed through the extrusion die into an implant channel (10) of the cannula device (4). The implant material solidifies in the implant channel, for example by means of cooling.

The invention relates to a cannula device and to a loading device for loading a cannula holder with an implant. The invention relates further to a method for loading the cannula device with an implant.

Implantation devices are used, for example, in the treatment of tumour patients and serve to administer a medicament subcutaneously to tumour patients. To that end, small rods of a biodegradable material, such as, for example, a plastics material, are produced as implants. The implant releases in the patient's body an active ingredient which, for example, causes a reduction in testosterone and accordingly inhibition of tumour growth. The implant is broken down, for example, by hydrolysis due to the natural water balance in the human body. The period for which the medicament is released in the patient's body typically extends over about four weeks.

The implantation device is used to insert the implant into the human body. The implantation device conventionally consists of three functional parts: an injection cannula, which contains the implant, a cannula holder, which can be permanently connected to the cannula or can comprise the cannula, and an applicator, which applies the implant from the injection cannula into the patient's tissue.

According to the prior art, an implant in the form of a small rod is inserted into the proximal opening of the cannula (the cannula tip) using tweezers and by means of a piston rod. In order to prevent the implant rod, which is thus loose in the cannula, from accidentally falling out, a solution of plastics material is then applied to the implant in the cannula by way of the proximal opening of the cannula, in order thus to fix the implant rod. The solution of plastics material can be introduced into the cannula in a predefined amount in the form of a drop by means of a dosing pipette. The drop then sits proximally in front of the implant rod and adheres both to the inside wall of the cannula and to the implant rod and cures at room temperature. It thus prevents the implant rod from falling out of the cannula.

If such an implant rod is to be applied, it is pressed against the cured plastics drop by means of a piston rod acting at the distal end of the implant rod, the plastics drop thereby becoming loose and releasing the implant rod.

That prior art has the disadvantage that several working steps are required in order to make a cannula with an implant rod ready for use. In addition, it is difficult to ensure a reproducible position of the plastics drop in the cannula. Finally, the surface of the implant rod, via which the active ingredient is to be released, is sealed by the plastics solution to differing degrees, so that parts of the surface are at times not available for the release of the active ingredient from the implant rod, which can result in a disadvantageous manner in uneven release of the active ingredient. Also, when the implant is applied, the plastics plug is injected at the same time, which can place additional stress on a patient.

It is an object of the present invention to find a solution in which the implant is fixed securely and reproducibly in the cannula and at the same time it is ensured that no additional material must be injected and that the properties of the implant are not adversely affected by additional material.

The object is achieved by the invention by the provision of an improved cannula device having an implant or for an implant, a loading device for loading a cannula device with an implant, and a method for loading a cannula device with an implant. According to the invention there are provided a cannula device according to claims 1 and 3, a loading device according to claim 10 and a method according to claim 12. Advantageous further developments of the invention are defined in the dependent claims.

A cannula device according to the invention with a proximal end and a distal end, which is provided for connection to an applicator, comprises an implant channel, which extends from the distal end to the proximal end of the cannula device, and an implant in the implant channel, wherein the implant is formed by solidification in the implant channel of a material introduced in a flowable state into the implant channel. An advantage of this cannula device is that the production of the implant and the loading of the cannula device with the implant take place in a single process and accordingly with an especially small number of process steps. As a result, the risk of error and the production costs are reduced. The cannula device preferably comprises a hose- or tube-like lining of at least a portion of the implant channel.

According to the invention, the implant is formed by solidification of a flowable material. The flowable material preferably comprises all the constituents of the implant that is to be produced. The flowable material is preferably a polymer melt which comprises all the constituents of the implant that is to be produced. The implant, and accordingly the flowable material, preferably comprises one or more active ingredients as well as a matrix, which is formed of biodegradable materials. There are preferably used as the matrix-forming material polymers, especially polylactide coglycerides and/or polylactides and/or lipids. Particular preference is given to poly-(lactide-co-glycolides) having an inherent viscosity of from 0.1 to 0.7 dl/g. Particular preference is given to polylactides having an inherent viscosity of from 0.1 to 0.7 dl/g. Particular preference is given to triglycerides, mono- and di-glycerides (of glycerol and fatty acids), wherein fatty acids are long-chained acids having 8 or more carbon atoms, preferably having from 8 to 30 carbon atoms. The fatty acids are preferably monocarboxylic acids having from 8 to 30 carbon atoms, which are saturated or mono- or poly-unsaturated and can optionally be branched. The fatty acids can also be partially replaced by acetic acids (acylated glycerides). Modified glycerides, such as, for example, PEGylated glycerides, can further be used. Phospholipids (e.g. lecithin) are also suitable. The active-ingredient-containing matrix melts above the pressure-dependent glass transition temperature. The mass preferably becomes flowable at temperatures of from 50 to 120° C., preferably from 70 to 90° C., under pressures of up to 20 kN, preferably from 0.5 to 2 kN. Melting of the mass and the glass transition temperature thereof are preferably measured by means of DSC (differential scanning calorimetry): In the case of phase changes such as melting or evaporation, temperature changes delta-T in comparison with a blank sample T_{Ref} occur. If the sample is heated in the measuring device, heat flows through the sample and the reference. If a sample changes during the measurement, for example by melting, a difference in the heat flow of the sample and the reference occurs, which is recorded by integration of the delta-T/T_{Ref} curve. The DSC method and suitable measuring devices are known in the specialist field.

The flowable material is preferably introduced into the implant channel under those conditions, for example by extrusion. Particularly preferably, the flowable material is extruded into the hose matrix at a temperature in the range of from 50 to 120° C. at a pressure of up to 20 kN.

According to the invention there can be used as active ingredients, for example, LHRH analogues, especially goserelin and/or leuprorelin.

In another embodiment, a cannula device with a proximal end and a distal end, which is provided for connection to an applicator, comprises an implant channel, which extends from the distal end to the proximal end of the cannula device, and a tube-like lining of at least a portion of the implant channel. Lining with a material which can be different from the material otherwise used for the cannula device allows the material properties to be adapted to the implant material and other boundary conditions. Optionally, the cannula device further comprises a piston rod, which is arranged in the implant channel and which preferably has a sealing piston which closes the implant channel at the location of the sealing piston. The piston rod permits particularly simple and at the same time accurate dimensioning of the implant, as is described below.

The lining of the implant channel is preferably formed by a tube or a hose, which is inserted into a bore in the cannula device. The implant channel preferably has an enlarged inside diameter in the region of the bore. The tube or hose preferably comprises a plastics polymer or copolymer. Preferred polymers are inert towards a reaction with the extrusion mass. Preferred plastics polymers are polyfluoropolymers, such as, for example, PFA, PTFA or FEP, especially polytetrafluoroethylene (e.g. Teflon). They can be used either on their own or together with other polymers, also in copolymerised form.

The material of the lining should be flexible in order to allow the implant to be removed after solidification, because the mass that is introduced can expand slightly after the extrusion pressure is removed. It is to be ensured that the injection unit has sufficient stability so that it can be handled easily by the user (doctor). The balance between the required resilient properties of the tube lining and the mechanical properties of the cannula device can be achieved by suitably choosing the materials for the device and the lining.

Particular preference is given to the combination of a cannula device of polycarbonate with an implant channel lining of Teflon. It has been found that the combination of polycarbonate and Teflon is especially stable to exposure to gamma radiation and heat, as act upon the cannula device during sterilisation, for example. The capacity for exposure to gamma radiation and heat was determined empirically. The combination of polycarbonate with an inserted Teflon hose was found to be particularly stable.

The piston rod (plunger) must be able to withstand the expected extrusion pressure of up to 20 kN and must ensure that the implant can readily be detached from the plunger after solidification of the extrusion mass. The piston rod (plunger) is preferably made of stainless steel, the adhesion properties of which can preferably be reduced by a suitable surface treatment, for example with silicone.

For insertion of the lining, the bore has an inside diameter and the tube has an outside diameter which are larger than the inside diameter of the implant channel. The tube can be rigid or flexible, that is to say hose-like. The bore and the tube can each have a smaller length than the implant channel, so that only a portion of the implant channel is lined with the tube, especially the portion that is filled by the implant or into which the implant is introduced. That portion is advantageously adjacent to the distal end of the cannula device. The inside diameter of a portion of the implant channel adjoining the bore is preferably smaller than the inside diameter of the bore and the outside diameter of the tube. The shoulder located in between prevents the tube from slipping during implantation.

The inside diameter of the hose- or tube-like lining is dependent upon the desired implant diameter, which is conventionally from 0.5 to 5 mm and preferably from 1 to 2 mm. The inside diameter of the lining preferably corresponds to the desired implant diameter. The diameter is preferably from 1 to 2 mm, more preferably from 1.2 to 1.8 mm and particularly preferably approximately 1.5 mm. The lower limit is determined by the mechanical stability of the implant. Implants having a diameter of less than 0.5 mm are generally not sufficiently stable. The upper limit is governed by the acceptability of the injection needle, the inside diameter of which must be larger than the diameter of the implant. The inside diameter of the injection needle is preferably from 0.1 to 0.5 mm larger than the diameter of the implant. Injection needles having an inside diameter of more than 5.5 mm cause large wounds and are not acceptable. The bore diameter is particularly preferably from 2 to 3 mm with, at the same time, a diameter of the implant channel of from 1 to 2 mm.

The length of the lining is preferably equal to or longer than the desired implant length. Preferred implant lengths are from 10 to 25 mm. The implant length will be established in dependence upon factors such as, for example, the desired use or dosage, the composition of the implant, the active ingredient concentration, etc. Accordingly, the lining length is preferably from 10 to 30 mm, more preferably from 20 to 30 mm and particularly preferably from 25 to 30 mm. The cannula device according to the invention can thus advantageously be used universally with different products.

The described cannula devices can be varied in many ways and optimised for different uses and boundary conditions. For example, the length of the implant can be smaller than the length of the implant channel. Furthermore, the implant channel can have a smaller inside diameter in the region of the implant than in other places. Those different inside diameters allow the implant to be held securely in its intended location during storage and transport and at the same time allow it to be moved easily on implantation.

The cannula device is preferably a cannula holder and can comprise a cannula which is connected to the cannula holder by a friction-based, interlocking or material-bonded connection. Alternatively, the cannula device is configured for connection to a cannula, for example by way of a luer or luer-lock connection. The implant channel preferably has a smaller inside diameter than the cannula at least in the region of the implant, or of an implant chamber provided to receive an implant. This has the advantages already mentioned. However, the implant chamber, in which the implant is formed or arranged, can also be located in the cannula, as a result of which an especially simple form of construction is achieved, with low production costs.

There can be used as the cannula a conventional cannula with the desired dimensions.

The cannula device preferably comprises a transparent material and/or a viewing window of transparent material. The viewing window is preferably arranged close to the proximal end of the cannula device, or close to the cannula. It preferably allows the implant channel to be observed in a viewing direction perpendicular to the implant channel. As a result, it is possible visually to check before implantation whether the cannula device is loaded with an implant and/or to observe the movement of the implant during implantation. The window can also be a cut-out portion in the cannula or be formed by the transparency of the cannula fastening in the region between the tube- or hose-like lining of the implant channel. This arrangement offers the advantage that, after the implant has solidified in the tube or hose, it can be displaced slightly so that it becomes visible in the window.

Both the material of the cannula device and the transparent material of the viewing window and the material of the sealing piston are each preferably pharmaceutically inert. Preferred materials are plastics, especially silicone, polycarbonate, as sold, for example, by Bayer under the trade name Makrolon, polypropylene, polyethylene and polytetrafluoroethylene. If a viewing window in the region of the needle holder is desired, polycarbonate is the preferred material therefor. For a viewing window in the region of the injection cannula, a cut-out portion is formed in the cannula. Non-transparent regions can, if desired, be obtained by the introduction of suitable non-transparent fillers. For the sealing piston required during production, a coating of silicone or polytetrafluoroethylene (Teflon) is preferably applied to a piston of optionally hardened stainless steel. The materials for the cannula device and insert must be heat-resistant up to the temperature at which the implant is extruded (from 50 to 120° C.). Furthermore, they must not become cloudy when exposed to gamma rays for sterilisation. It has been found that these requirements are met by commercially available materials which, as regards their monomer and/or additive composition, comply with the legal requirements for allowable plastics, such as, for example, EU directive 2002/72/EC relating to “Plastic materials and articles intended to come into contact with foodstuffs”, the German “commodities act”, the American FDA-modified ISO 10993-1 “Biological Evaluation of Medical Devices”, the European Pharmacopoeia (4th supplement 2002, 3.2.2), the US Pharmacopeia XXII Class VI (biocompatibility) or the like. A preferred material is a polycarbonate which is obtainable under the name “Makrolon 2858”. Bayer, e.g. colourless (Makrolon 2858 550115) and meets the legal requirements according to material specification and safety certificate.

The implant preferably comprises a plastics material which contains or is mixed with a pharmaceutical active ingredient. Particularly preferably, the implant comprises a biodegradable plastics material. Particular preference is given to biodegradable plastics materials which are broken down by hydrolysis due to the natural water balance in the human body, after the implant has been injected, preferably subcutaneously, into a patient. Preferred examples of biodegradable plastics materials include polymers and copolymers of lactic acid and/or glycolic acid, especially polylactides and poly-lactide-co-glycolides. Also preferred are polyesters and lipids. Particularly preferred implants comprise a plastics material/active ingredient combination of poly-lactide-co-glycolide and goserelin.

The proximal end of the cannula device preferably has a cannula coupling for connecting the proximal end of the cannula device to the cannula by a friction-based, material-bonded or interlocking connection. The distal end of the cannula device preferably has a cannula coupling for connecting the distal end of the cannula device to an applicator by a friction-based, material-bonded or interlocking connection.

At the distal end of the cannula device there is particularly preferably used a modification of a conventional luer-lock coupling, in which the counter-piece of the luer-lock attached to the applicator, unlike a conventional luer-lock, does not have an inner cone. Penetration of the cone into the distal end of the cannula holder is not possible in the device of the present invention because the extruder die must be able to form a tight seal with the cannula holder during the extrusion.

A loading device for loading an implant channel of a cannula device with an implant comprises an extrusion die for extruding a flowable implant material and a sealing surface on the extrusion die. A holding device holds a cannula device at the extrusion die in such a manner that the cannula device is in contact with the sealing surface. A material feed device is provided for feeding the flowable implant material through the extrusion die into the implant channel, the implant material preferably being fed free of air bubbles. The loading device preferably further comprises a stop for a piston rod, which is arranged in an implant channel of a cannula device held at the extrusion die by the holding device and is movable along the implant channel. The length and accordingly the mass of the implant are determined in an especially simple and reliable manner by the stop.

The feed and positioning device preferably consists of optionally hardened stainless steel, the surfaces that come into contact with the extrusion mass optionally being coated with an inert coating of silicone or polytetrafluoroethylene (Teflon) in order to prevent adhesion to the extrusion mass and/or undesirable interaction with any aggressive or corrosive constituents thereof. There can be used as the piston rod a sealing piston as described above. It is preferably made of optionally hardened stainless steel, likewise with an optional coating of silicone or polytetrafluoroethylene (Teflon).

Any conventional extruder can be used for the extrusion, such as, for example, a 1-screw extruder, a 2-screw extruder, a piston extruder or the like.

A method for loading a cannula device comprises the following steps: holding the cannula device at an extrusion die so that the cannula device is in contact with a sealing surface of the extrusion die; feeding a flowable implant material through the extrusion die into an implant channel of the cannula device; solidifying the implant material in the implant channel. Solidification is effected, especially in the case of a thermoplastic implant material, preferably by cooling or by polymerisation. The cannula device can be separated from the extrusion die before or after the solidification. This method combines the two steps of producing the implant and loading the cannula device with the implant. As a result, the method is particularly simple, quick and reliable. In particular, it is not necessary to prepare and clean a mould for the implant, to handle the implant after its production and to insert the implant into the cannula device. The risk of loss, damage or contamination of the implant is minimised as a result.

The parameters of the individual production steps will be determined in dependence upon the extrusion mass. Conventional process steps in the production of implants comprise cold pre-compression at from 1000 to 5000 N for from 10 to 30 minutes followed by warm pre-compression at from 70 to 120° C. and from 200 to 1000 N for from 10 to 60 minutes and extrusion at from 70 to 120° C. and from 100 to 2000 N. Further conditions of the individual process steps can be found, for example, in WO2007/107328 A1 (p. 10-11).

Before the flowable implant material is fed in, a piston rod is preferably introduced into the implant channel. The piston rod is displaced out of the implant channel when the implant material is fed in. The piston rod preferably has a sealing piston which closes the implant channel at its location. The implant material is preferably fed until the piston rod abuts a stop. The piston rod allows the length and accordingly the mass of the implant to be determined in an especially simple and reliable manner.

Furthermore, before the flowable implant material is fed in, and especially also before the cannula device is held at or connected to the extrusion die, a tube is preferably inserted into a bore in the cannula device. In order to prevent the implant from adhering to the cannula device or the hose, the implant is preferably moved after the implant material has solidified or partially solidified, without thereby leaving the cannula device.

Preferred exemplary embodiments of the present invention are explained in detail below by means of the accompanying figures.

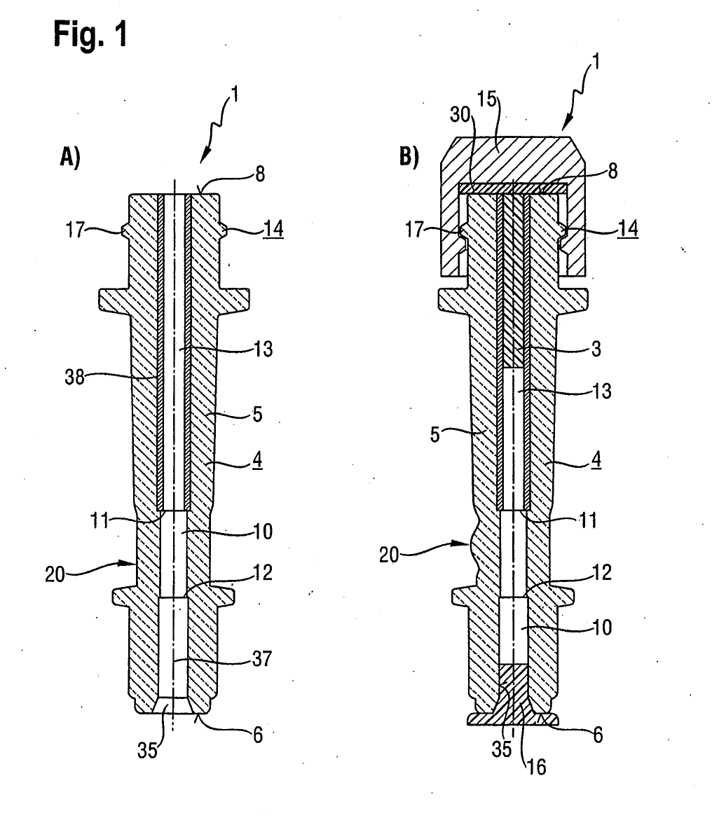

FIG. 1 shows in schematic views A) and B) cross-sections through an implantation device according to a first embodiment of the invention;

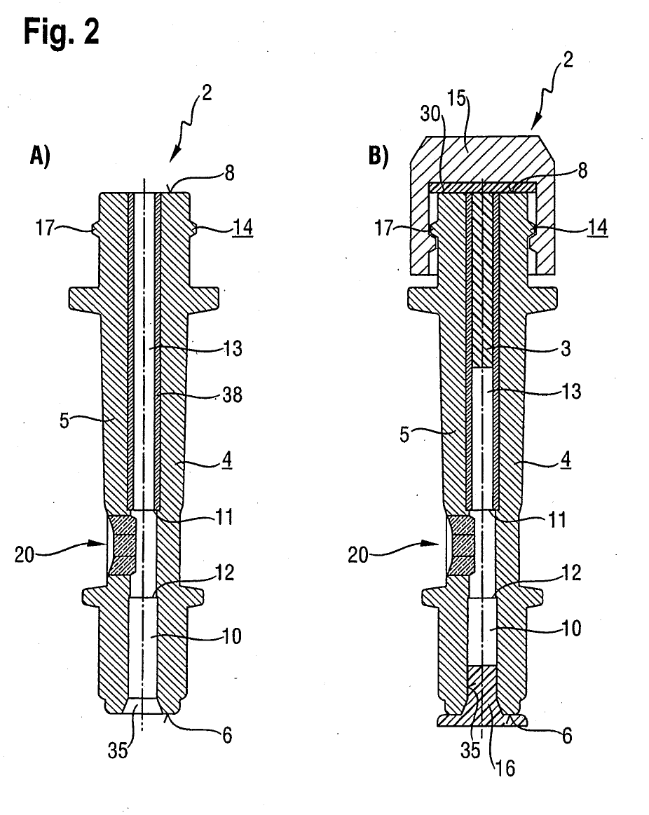

FIG. 2 shows in schematic views A) and B) cross-sections through an implantation device according to a second embodiment of the invention;

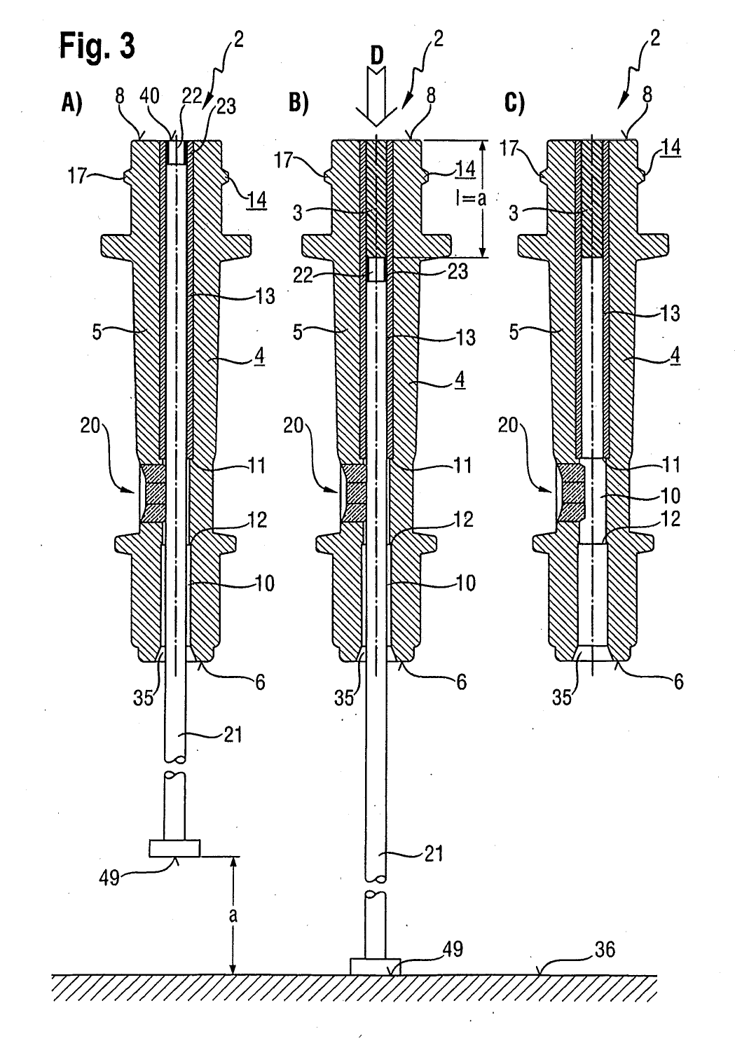

FIG. 3 shows in schematic views A), B) and C) the loading of a cannula holder with an implant;

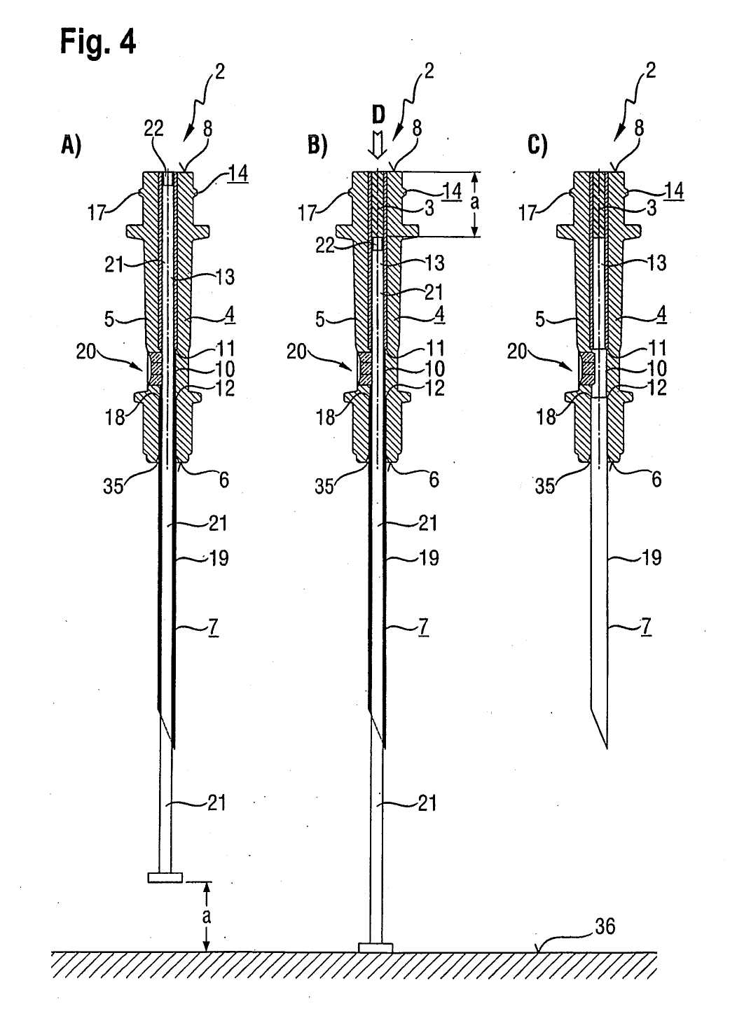

FIG. 4 shows in schematic views A), B) and C) the loading of an implantation device with an implant;

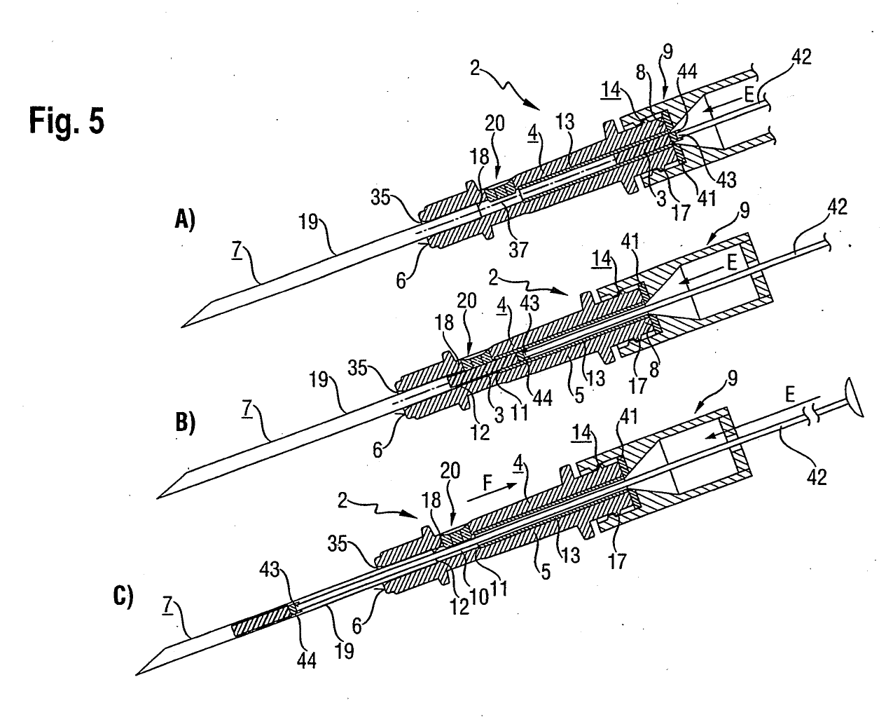

FIG. 5 shows in schematic views A), B) and C) an implantation device on application of the implant;

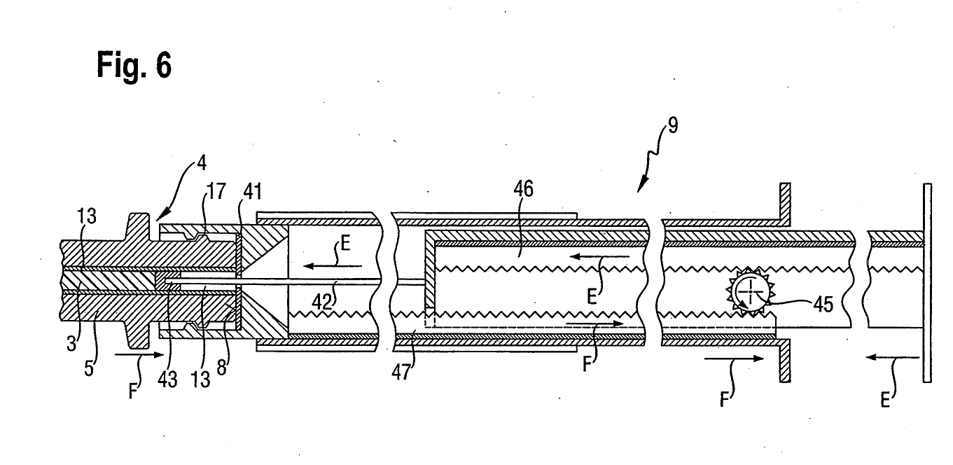

FIG. 6 shows a schematic diagram of a novel applicator;

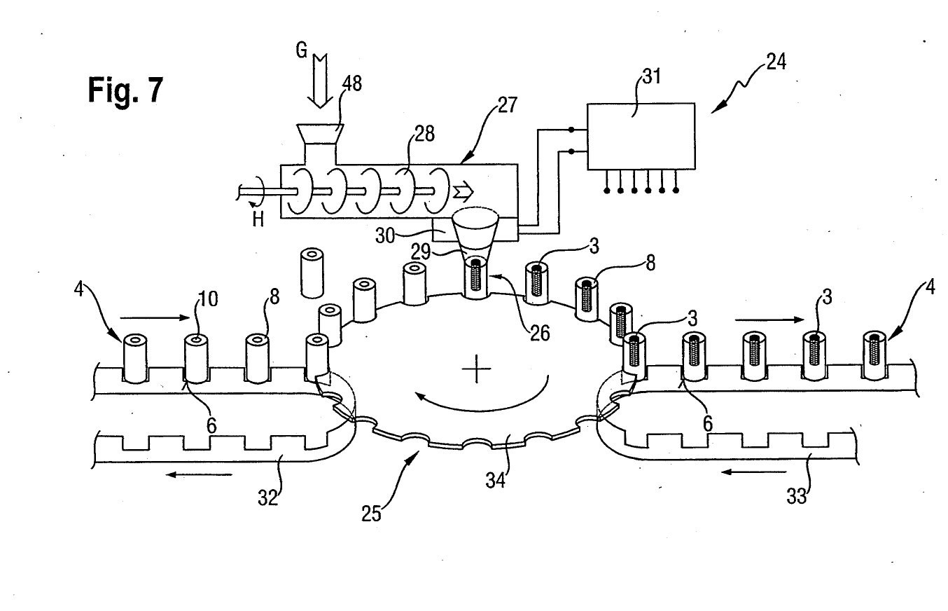

FIG. 7 shows a partial perspective schematic diagram of a loading device.

FIG. 1 shows in schematic views A) and B) cross-sections through an implantation device 1 according to a first embodiment of the invention. FIG. 1 A) shows a cannula device 4, especially a cannula holder, which has an implantation device 1 together with a housing 5 with a proximal end 6 and a distal end 8. Arranged along a cannula holder axis 37 is a central channel, which forms an implant channel 10 and has a first shoulder 11 and a second shoulder 12, the channel cross-section being reduced between the shoulders 11 and 12. The shoulder 12 serves as a stop for a cannula in the form of an injection needle, which can be inserted as far as the shoulder 12 by way of a proximal opening 35 of the cannula holder 4 and can there be connected to the cannula device 4 preferably by a material-bonded, friction-based or interlocking connection.

The shoulder 11 delimits the region of an implant chamber 13, which in this embodiment of the invention is formed by a Teflon tube or hose section 38 and in turn forms a portion of the implant channel. The Teflon tube or pipe section is arranged in a bore of the cannula device 4. An implant material, preferably an active ingredient/polymer mixture, can be extruded directly into the implant chamber 13. The weight of such an implant in the implant chamber 13, and accordingly also the active ingredient content in the case of a fixed active ingredient concentration in the mixture, is determined by the inside diameter of the hose section 38 and by the length of the hose section 38 that is filled with an implant material. Depending upon the embodiment of the implantation device, the hose diameter for the implant chamber 13 can be varied.

The length of the implant is fixed by a piston rod having a sealing piston. The piston rod is inserted from one end, preferably from the proximal opening 35, with the sealing piston first, into the implant channel until the sealing piston is flush with the other end of the implant channel, preferably the distal opening. When the implant chamber is filled with the implant material, the sealing piston, and accordingly the piston rod as a whole, is displaced from or pushed out of the implant channel until the piston rod abuts a stop. The length of the implant is accordingly fixed by the arrangement of the stop.

The hose section 38, and accordingly the implant chamber, extend from the distal end 8 of the cannula holder 4 to the first shoulder 11, at which the cross-section of the implant channel 10 is narrowed. In that transition region from the implant chamber 13 to the cannula arranged in the proximal opening 35 there is provided a viewing window 20, which allows a view into the implant chamber in a viewing direction perpendicular thereto. In the embodiment of FIG. 1 A), the viewing window 20 is simply a smooth or polished outer surface of the housing 5, which in this embodiment is made of a transparent plastics material.

Instead of inserting a cannula in the form of an injection needle into the proximal opening 35, it is also possible to provide on the outer periphery of the cannula holder 4 an additional coupling device for releasably connecting a cannula to the proximal end of the cannula holder 4.

On the outer surface of the cannula holder 4 in the region of the distal end 8 there is provided a coupling device 14 in the form of a luer-lock external thread 17, which is used on the one hand for attaching a cap in a media-tight manner after the implant chamber 13 has been filled with an implant and on the other hand for attaching an applicator to the distal end 8 of the cannula holder for application of the implant.

FIG. 1 B) shows a schematic view of a cross-section of the cannula holder 4 shown in FIG. 1 A) with an implant 3 introduced into the implant chamber 13, a cap 15 with a luer-lock internal thread having been screwed onto the distal end 8 of the cannula holder 4 so that a gasket 39 in the cap 15 closes the distal end 8 in a media-tight manner. The proximal end 6 of the cannula holder 4 can be closed in a media-tight manner by means of a suitable stopper 16. In the region of the viewing window 20, a concave contour is ground into the transparent housing 5 in order to permit a lens effect for observation during application of the implant.

FIG. 2 shows in schematic views A) and B) cross-sections through an implantation device 2 according to a second embodiment of the invention. Components of FIG. 2 having the same functions as in FIG. 1 are labelled with the same reference numerals and are not discussed further. The difference with respect to the first embodiment of the invention is that the housing 5 of the cannula holder 4 according to FIG. 2A) is made of an optically non-transparent material. In this case, an opening has been formed in the optically non-transparent material in the region of the viewing window 20, which opening is filled with a transparent plastics material. It is thus ensured that, here too, it is possible to observe the movement of the implant during application in the transition region between the two shoulders 11 and 12.

FIG. 2 B) shows this second embodiment of the invention with an implant 3, which is arranged in the implant chamber 13, the cannula holder 4 being closed in a media-tight manner during storage and transport by means of a cap 15, as is already known from FIG. 1B), and a stopper 16.

FIG. 3 shows in schematic views A), B) and C) the loading of a cannula holder 4 with an implant 3. To that end, as is shown in FIG. 3 A), a piston rod 21 is inserted into the implant channel 10 of the cannula holder from the proximal opening 35 of the cannula holder 4. To that end, the piston rod 21 has at its distal end 40 a sealing piston 22, which can be coated with a medicinally inert material. The coating 23 ensures that the implant material that is to be introduced is not contaminated. The material of the coating 23 is preferably likewise Teflon. The piston rod 21 is pushed through the implant channel 10 and especially through the implant chamber 13 until the distal end 40 of the piston rod 21, and accordingly the sealing piston 22, is flush with the distal end 8 of the cannula holder 4. The distal end 8 of the cannula holder has a sealing surface which is preferably flat, so that the distal end 8 can be positioned at an extrusion die in a media-tight manner. There is additionally provided a stop 36 which can be set at a distance a from the proximal end 49 of the piston rod 21 in order to specify the length I of an implant 3 in the implant chamber 13.

In FIG. 3 B), the cannula holder 4 with the piston rod 21 is located at an extrusion die (not shown), which is positioned in a media-tight manner at the distal end 8 of the cannula holder 4. A flowable implant material, which preferably comprises a mixture of an active ingredient and a polymer, is extruded directly into the implant chamber 13 in the direction indicated by arrow D. The cross-section of the implant chamber 13 is smaller than the inside cross-section of the transition region between the shoulders 11 and 12 and also smaller than the inside cross-section of the cannula that is to be positioned. When the active ingredient/polymer mixture is brought into a loading position of an automatic loading device, the piston rod 21 is displaced from or pushed out of the proximal opening 35 of the piston holder 4 until the stop 36 has reached the loading position through the proximal end 49 of the piston rod 21. Accordingly, the length I of the implant in the implant chamber is given by the distance a.

FIG. 3 C shows the cannula holder 4 with an implant 3 in the region of the distal end 8 of the implant chamber 13. The cannula holder 4 can then be sterilised by means of irradiation, preferably by gamma rays, and packaged and supplied in sterile form. To that end, as is shown in FIG. 1 B) and FIG. 2 B), a cap can be placed in a media-tight manner on the distal end 8 and a stopper can be placed on the proximal end 6 of the cannula holder 4.

FIG. 4 shows in schematic views A), B) and C) the loading of an implantation device 2 with an implant. Unlike in FIG. 3, in the case of this loading the cannulas 7 are already arranged with their distal end 18 in the proximal opening 35 of the cannula holder 4, the distal end 18 abutting the shoulder 12 of the cannula holder 4. In order to define the length of the implant material that is to be introduced there is provided a piston rod 21 which is longer than the piston rod shown in FIG. 3 by the length of the cannula 7. Components in FIGS. 4 A), B) and C) which have the same functions as in FIG. 3 are labelled with the same reference numerals and are not discussed further. Accordingly, FIG. 4 merely demonstrates that it is also possible to load the implant chamber 13 with a predetermined amount of active ingredient/polymer mixture with the cannula 7 already inserted. Sterilisation can then again be carried out, and a cannula holder 4 loaded with an implant 3 and with an attached cannula 7 can be supplied.

Alternatively to loading the cannula holder from its distal end, the cannula holder can also be loaded with an implant from its proximal end, especially when the cannula holder is not provided with the cannula until after loading.

FIG. 5 shows in schematic views A), B) and C) an implantation device 2 on application of the implant 3. To that end, as is shown in FIG. 5 A), an applicator 9 is attached to the distal end 8 by means of the coupling device 14, the mouthpiece of the applicator having a disk-like seal 41 which connects the distal end 8 of the cannula holder 4 to the applicator 9 in a media-tight manner. The mouthpiece of the applicator 9 preferably has a luer-lock internal thread, which engages in a luer-lock external thread 17 of the cannula holder 4. A central applicator rod 42 is brought into a coaxial position relative to the cannula holder axis 37 and is in contact with the distal end of the implant 3 with a sealing piston 43, which has a medicinally inert coating 44.

In FIG. 5 B), a pressure is exerted on the implant 3 in the direction indicated by arrow E, so that the implant 3 is pushed out of the implant chamber 13 and guided past the viewing window 20, whereby monitoring by the personnel carrying out the application is possible.

In FIG. 5 C), the implant 3 has been introduced into the injection needle 19 in the direction indicated by arrow E by means of the applicator rod 42 and, if the injection needle 19 is already arranged subcutaneously, can finally be pushed out of the injection needle 19 subcutaneously and positioned. To that end, a simultaneous movement of the injection needle 19 in the distal direction is advantageous in order to position the implant 3 subcutaneously.

FIG. 6 shows a schematic diagram of a novel applicator 9, which has a gear wheel 45. This couples two toothed rods 46 and 47 together and has the effect that, when the implant 3 is pushed in direction E, a withdrawal of the injection needle in direction F takes place synchronously. However, before that synchronous movement takes place, the implant 3 is brought beyond the position shown in FIG. 5 C) to the proximal end of the cannula 7.

FIG. 7 shows a partially perspective schematic diagram of a loading device 24 for loading cannula holders 4 with an implant 3. To that end, the loading device 24 has a feed device 25, which in this embodiment of the invention has two conveyor belts 32 and 33 as well as a rotary plate 34 and an extruder 27 having a mixing device 28. The entire loading device 24 is controlled by a control and monitoring device 31. A polymer granulate and the active ingredient are introduced into the opening 48 of the extruder 27 in the direction indicated by arrow G and are brought into a mixing device 28 by way of a screw drive by rotation in the direction indicated by arrow H, a pressure at the same time building up, which pushes the mixture of active ingredient and polymer granulate to an extrusion die 29. A heater 30 heats the extrusion die 29 to a softening temperature of the polymer granulate, so that it becomes liquid or at least viscous, that is to say flowable under pressure.

The cannula holders 4 with empty, unfilled implant chambers are fed by way of a feed conveyor belt 32 to the rotary plate 34, which transports them cyclically into a loading position 26 beneath the extrusion die 29. By opening and closing of the extrusion die 29, an amount of implant material set beforehand by means of a piston rod is introduced into implant chambers of the cannula holders 4 in the loading position 26.

The cannula holders 4 loaded or filled with implant material are provided at their distal ends 8 with caps shown in FIGS. 1B) and 2B) and are sealed at their proximal ends 6 with corresponding stoppers and fed to a take-off conveyor belt 33. The conveyor belts 32 and 33 can be in the form of endless belts and are loaded and emptied by an automatic unit (not shown here), Sterilisation positions can be provided on the removal conveyor belt 33, before the filled or loaded cannula holders 4 are removed from the take-off conveyor belt 33 and dispatched packaged in a sterile manner.

List of Reference Numerals

1 Implantation device (first embodiment)

2 Implantation device (second embodiment)

3 Implant

4 Cannula holder

5 Housing of the cannula holder

6 Proximal end of the cannula holder

7 Cannula

8 Distal end of the cannula holder

9 Applicator

10 Implant channel

11 Shoulder in the channel

12 Shoulder in the channel

13 Implant chamber

14 Coupling device

15 Cap

16 Stopper

17 Luer-lock external thread

18 Distal end of the cannula

19 Injection needle

20 Viewing window

21 Piston rod

22 Sealing piston

23 Coating of the sealing piston

24 Loading device

25 Feed device

26 Loading position

27 Extruder

28 Mixing device

29 Extrusion die

30 Heater

31 Control and monitoring device

32 Conveyor belt

33 Conveyor belt

34 Rotary plate

35 Proximal opening of the cannula holder

36 Stop

37 Cannula holder axis

38 Tube

39 Gasket

40 Distal end of the piston rod

41 Seal

42 Applicator rod

43 Sealing piston

44 Coating

45 Gear wheel

46 Toothed rod

47 Toothed rod

48 Opening

49 Proximal end of the piston rod

a Distance

D Direction of arrow

E Direction of arrow

F Direction of arrow

G Direction of arrow

H Direction of arrow

I Length of the implant rod

1-15. (canceled)

16. A cannula device comprising
(a) a proximal end;
(b) a distal end, which is provided for connection to an applicator;
(c) an implant channel, which extends from the distal end to the proximal end of the cannula device; and
(d) an implant in the implant channel, wherein the implant is formed by introducing implant material, in a flowable state, into a region of the implant channel and solidifying the implant material.

17. The cannula device of claim 16, further comprising a hose- or tube-like lining of at least a portion of the implant channel.

18. The cannula device of claim 17, further comprising a bore in the cannula device, wherein said bore has an inside diameter which is larger than the inside diameter of the implant channel, wherein the lining is formed by a tube inserted into the bore and the inside space of said tube forms at least a portion of the implant channel.

19. The cannula device of claim 18, wherein the bore starts at one end of the cannula device, and the bore and the tube each have a shorter length than the implant channel.

20. The cannula device of claim 17, wherein the implant channel has a smaller inside diameter in the region of the implant than in other places.

21. The cannula device of claim 17, further comprising a cannula, wherein the implant channel has a smaller inside diameter than the cannula at least in the region of the implant.

22. The cannula device of claim 17, wherein the cannula device has a transparent material or a viewing window of transparent material.

23. The cannula device of claim 17, wherein the implant comprises goserelin, leuprorelin, or a combination thereof, as active ingredient.

24. A cannula device comprising
(a) a proximal end;
(b) a distal end, which is provided for connection to an applicator;
(c) an implant channel, which extends from the distal end to the proximal end of the cannula device; and
(d) a tube-like lining of at least a portion of the implant channel.

25. The cannula device of claim 24, further comprising a bore in the cannula device, wherein said bore has an inside diameter which is larger than the inside diameter of the implant channel, wherein the lining is formed by a tube inserted into the bore and the inside space of said tube forms at least a portion of the implant channel.

26. The cannula device of claim 25, wherein the bore starts at one end of the cannula device, and the bore and the tube each have a shorter length than the implant channel.

27. The cannula device of claim 24, further comprising an implant formed by introducing implant material, in a flowable state, into a region of the implant channel and solidifying the implant material.

28. The cannula device of claim 27, wherein the implant channel has a smaller inside diameter in the region of the implant than in other places.

29. The cannula device of claim 27, further comprising a cannula, wherein the implant channel has a smaller inside diameter than the cannula at least in the region of the implant.

30. The cannula device of claim 24, wherein the cannula device has a transparent material or a viewing window of transparent material.

31. The cannula device of claim 24, wherein the implant comprises goserelin, leuprorelin, or a combination thereof, as active ingredient.

32. A loading device for loading an implant channel of a cannula device with an implant comprising
(a) an extrusion die for extruding a flowable implant material;
(b) a sealing surface on the extrusion die;
(c) a holding device for holding a cannula device at the extrusion die in such a manner that the cannula device is in contact with the sealing surface; and
(d) a material feed device for feeding the flowable implant material through the extrusion die into the implant channel.

33. The loading device of claim 32, further comprising a stop for a piston rod, which is arranged in an implant channel of a cannula device held at the extrusion die by the holding device, wherein said piston rod is movable along the implant channel.

34. A method for loading a cannula device with an implant comprising
(a) holding a cannula device at an extrusion die in such a manner that the cannula device is in contact with a sealing surface of the extrusion die;
(b) feeding a flowable implant material through the extrusion die into an implant channel of the cannula device; and
(c) solidifying the implant material in the implant channel.

35. The method of claim 34, wherein a piston rod is introduced into the implant channel before the flowable implant material is fed in, and wherein the piston rod is displaced at least partly out of the implant channel when the implant material is fed in.

36. The method of claim 34, wherein the cannula device has a bore, the inside diameter of which is larger than an inside diameter of the implant channel, and wherein, before the flowable implant material is fed in, a tube is inserted into the bore, the inside space of which tube forms part of the implant channel.

37. The method of claim 34, wherein the flowable implant material comprises goserelin, leuprorelin, or a combination thereof, as active ingredient.
